(19) United States
(12) Patent Application Publication
(10) Pub. No.: US 2020/0085673 A1
(43) Pub. Date: Mar. 19, 2020

(54) METHOD AND SYSTEM FOR CUSTOMIZED OPERATION OF A THERAPEUTIC DEVICE

(71) Applicant: Golden GM Holdings SDN. BHD., Iskandar Puteri (MY)

(72) Inventors: Jung Seo, Duluth, GA (US); William P. Yi, Cumming, GA (US); Michael K. Lee, Duluth, GA (US)

(21) Appl. No.: 16/135,209

(22) Filed: Sep. 19, 2018

(51) Int. Cl. A61H 15/00; G16H 20/30; A61H 7/00

(52) U.S. Cl. CPC A61H 15/0078; G06F 3/011; A61H 7/007; G16H 20/30

(57) ABSTRACT

A therapeutic device includes sensors for collecting biometric health-related parameter information, such as a person's vital signs, during a therapy session for a particular user. A therapeutic program includes an artificial intelligence learning algorithm that uses the biometric health-related information for a particular user acquired during one or more training periods to customize therapeutic parameter values of a therapeutic program to deliver a specialized massage/therapy session that minimizes detriment, and maximizes benefit, to a user by comparing biometric health-related information collected during a therapy session to criteria that corresponds to the health-related information. Biometric health-related information may be stored to a database either local or remote relative to the therapeutic device. Users may retrieve biometric health-related information associated with therapy sessions and view graphical comparisons of changes to their vital signs and other collected data relative to changes in therapeutic parameter values as modified by the therapeutic program.

### FIELD

The field relates, generally, to Internet of Things systems and devices and, more particularly, to a system and method for operating a therapeutic device in a particular manner that is customized for a particular patient.

### BACKGROUND

Therapeutic devices, such as massage chairs, whirlpool tubs, saunas, heating blankets, and the like, provide relaxation, comfort, and sometimes medical relief to users. A therapeutic massage chair, which may be referred to herein as simply a massage chair, may provide massage action to various parts of a user's body, including various parts of the back, torso front and sides, arms, legs, neck, scalp, fingers, toes, feet, hands, knees, shoulder, hips, etc. A massage chair may provide such massage action by activating one or more particular motors corresponding to a particular part of the body to be massaged, and the motor may cause a massaging component to operate, such as an eccentric weight on the end of the motor's shaft, a reciprocating motion, one or more rollers, one or more roller balls, one or more air bladders that may be actuated by an air pump or a compressed air reservoir, or by activating other forms of massage. A massage chair may also provide heating and cooling to certain parts of the body of a user who uses the massage chair. For example, heating elements may provide heat to an area with sore muscles, or a cooling component, such as a Peltier device (or other type of cooling device), may cool an area of the user's body that is experiencing inflammation.

Massage chairs typically include an electronic control processing module, which may include a digital signal processor or a general microprocessor that causes various massaging components to operate based on a predetermined session operation program selected from a plurality of predetermined session operation programs. For example, a user may select a massage session program that only causes a lower back massage component to operate vigorously if only the user's lower back feels tense. Or, for overall relaxation a user may select a program that causes gentle massaging, along with heating or cooling, of all areas or his, or her, body, following a stressful situation such as a long drive or participation in an athletic event. A microprocessor of a massage chair's control module may cause a screen that is coupled to the control module to display a user interface for receiving massage program selection from a user. Upon selection of a therapeutic/massage program, the user may sit in the massage chair and enjoy a massage according to the program that he, or she, selected from the user interface.

Available to the retail market beginning in the 1980's, massage chairs are finally becoming one of the most popular home therapy products on the market. These chairs were developed to provide the benefits of massage therapy to the home user on a regular basis. Also, by being placed in one's home, it affords a great deal of convenience.

Massage therapy has been used for centuries to treat pain and help with relaxation. Massage is also credited with boosting the body's immune system through the stimulation of reticular cells. Like a traditional massage, the massage chair is designed to reduce tension, reduce headaches, relax muscles, and alleviate pain. Massage chairs are thought to help sufferers of digestive problems, circulation problems, and chronic fatigue.

Earlier models of massage chairs were not very effective. But as technology developed, the mechanisms became more realistic and reliable. While a massage chair may not be able to simulate the total therapeutic benefits of a massage by hand, at the very least it provides massage motions that are consistent. Some people even say that the electronic massage is in fact better as the motions never tire and they are delivered in a rhythmic and regular fashion.

Current massage chairs may be programmed via digital computer programs to provide differing massage actions during a therapy session. Programs may pinpoint problem areas and allow choice of firmness; choice of massaging mechanisms and actions of a chair may vary. Some feature popular shiatsu style kneading action, while others employ vibration and rollers to achieve that “ooh, ahh” sensation.

If an individual has an injury like a pulled muscle or strained back, immediate treatment may include taking pain relievers, applying ice, and avoiding strain for a day or two. On-going pain may indicate other health conditions like arthritis or cervical disc problems, which should be evaluated by a physician. Treatment may include massage to relieve muscle spasm and pain.

Massage helps to soothe aching muscles, relax the body, and relieve tension. Some forms of massage are also believed to provide health benefits such as improving digestion, boosting the immune system, relieving headaches, stimulating circulation and controlling minor depression.

However, current massage chairs fail to deliver personalized massage that truly caters to the user's wellbeing. Users do not have knowledge as to how to maximize use of a massage chair correctly. Users may use pre-installed massage options of a massage therapy session program, but current programs may not provide an optimal and efficient massage because the massage program that operates a massage chair, or the user, do not know what kind of massage the user needs to provide the most medical/therapeutic benefit to the user. Current massage chairs do not receive a user's biometric parameter information during a therapy session that is used to customize coefficients, parameter values, functions, or other values that a massage program uses to control operation of a massage chair, or other therapeutic device.

### SUMMARY

A therapeutic device, such as a massage chair may have one or more massage component that can be manipulated by one or more corresponding massage actuators. For example, a back part or a seat part of a massage chair may include corresponding massage actuators can move, deform, manipulate, otherwise operate the corresponding massage one or more massage components for massage purposes. The massage actuators may include motors, heating elements, cooling elements, compression bands, pumps, valves, and the like.

Biometric parameter information that may be indicative of a particular user's health status may be collected by monitoring and storing information contained in sensor signals during a therapy session. The health/biometric parameter information collecting function may be facilitated by one or more sensors built into an armrest of the massage chair as well as being connected or coupled (perhaps wired or wirelessly) to other locations of therapeutic device, such as a massage chair, and providing a personalized massage according to a custom program that caters to a particular user's health state as determined by the information collected from the sensors' signals. The health/biometric parameter information collecting function may be facilitated by one or more sensors built into a user's user device, such as a smart phone or smart watch.

An automated health collection system uses biometric parameter information collected from a user during a therapy session where the user uses a therapeutic device during the therapy session. In an aspect, an interface is provided that allows a user to check their health vital signs and other biometric parameter information such as heart rate, oxygen saturation level, glucose level, blood pressure, stress level, skin moisture, electrocardiogram information, electromyogram information, breathing rate, breathing depth (i.e., amount of air inhaled per breath), electrodermal activity information, and body temperature. In another aspect, biometric parameter information that has been collected from sensors connected to a user during a therapy session may be provided to a control module of the chairs, which may be a central computing system of the chair. The collected biometric parameter information may be used to optimize a program that controls a massage, or other type of therapy session, based on the collected health data/biometric parameter information. In another aspect, users may track their health data and type of massage used through an application running on a platform such as a smart phone, smart watch, tablet, or PC. A user may send collected biometric parameter information data to a medical institution of their choice for research or examination.

A sensor used to collect biometric parameter information may include a fingertip sensor that detects pulse rate (heart rate), oxygen saturation level, and glucose level; or a wrist/upper arm sensor that measures blood pressure, skin moisture, skin temperature, and other similar vital signs. These sensors may be located on the top of the massage chair and can be readily accessed by a user when sitting down on the massage chair. Other sensors may include sensors that attach to a user's chest to measure breathing parameters and heart parameters, such as may be collected during an electrocardiogram test, or sensors that are placed on or around a user's head to measure brain activity.

The collected biometric parameter information data may be sent to the massage chair's central computer system. A therapy program, which may be referred to herein as a therapeutic program, running on the chair's computer system may calculate an optimized massage based on received biometric parameter information by modifying therapeutic parameter values that the program uses to control various therapeutic components, such as massage components, temperature components (heating or cooling), rolling components, kneading components, vibration components, and the like. The biometric parameter information may be received from sensors during a therapy session that uses default therapeutic parameter values. Based on changes in biometric parameter information, and comparison of such information to criteria that may be specific to a particular user, the therapy program may modify values used by the program to fine-tune massage options and operation of one or more therapeutic components that is best suited for the user and their current health. For example, if a sensor, such as a fingertip sensor, detects an irregular heart rate, the massage chair's control module processor may recommend a massage option that is suited to relieve irregular heart beat and may modify therapeutic parameter values such that the program operates according to such recommendation.

In an aspect, a user may view or track health data collected before and after a massage and may view massage options implemented during a massage, such as massage duration, massage intensity, massage temperature, massage frequency, and the like, for one or more massage components, through the system's platform via the user's smartphone and/or PC. The collected data may be securely stored (i.e., may be accessed by providing authentication credentials) in a database and the user may opt in to share private information to medical institutions for examination purposes or to a research institute for personal gain. Reports may be generated that graphically show a relationship between application of therapeutic parameter values to operation of a massage chair/device and biometric sensor information with respect to time.

A benefit is that collected biometric parameter information may be used to modify values of a therapy program to customize the program to a particular user's medical and health needs in response to a massage. Furthermore, because the modification of therapy program values, such as therapeutic parameter values, coefficients, factors, or other values may be automatic, a particular user need not understand what preset massage option is best for their body, and thus a user does not need to manually enter therapeutic parameter values into a an interface that provides the values to the program. In addition, use of collected sensor data may proactively aid in identifying medical issues that may facilitate seeking early intervention and avoiding hospitalization.

In an aspect, a system comprises a therapeutic device that includes one or more therapy-delivering components operated by one or more corresponding therapy-delivering component actuators. The actuators may be motors, electrical supplies, air supplies, water supplies, and the like that cause massaging components, such as massaging components or heating/cooling elements, respectively, of a massage chair to deliver therapeutic/massaging action.

The therapy-delivering components may be included in a massage chair and may deliver massaging/therapeutic action to one or more of a user's head, neck, shoulder(s), upper arm(s), lower arm(s), hand(s), foot or feet, torso, upper leg(s), lower leg(s), upper back, middle back, lower back, or other body area.

The system may also comprise one or more biometric parameter sensors and a control module communicatively coupled with the one or more therapy-delivering component actuators and communicatively coupled with the one or more biometric parameter sensors. The control module may include circuitry for receiving signals from the biometric parameter sensors and for converting the signals to a format suitable for processing by one or more digital signal processors (“DSP”). The control module may also include circuitry for receiving therapeutic parameter values and for generating control signals to therapy-delivering components based on the received therapeutic parameter values such that operation of the therapy-delivering components corresponds to the received therapeutic parameter values. The one or more DSPs, or other processors such as one or more general application microprocessors, may be located in the control module, or some or all of the processors may be located remote from the control module, such as in a user's smart phone or within a server that is accessible via an internet protocol communications link, such as the world wide web or a wired or wireless local area network.

The biometric parameter sensors may include a heart rate sensor, a blood oxygen saturation percentage determining sensor, a body temperature sensor, a stress level sensor, a skin moisture sensor, a blood pressure sensor (for example, a blood pressure cuff), an electrocardiogram sensor, an electromyogram sensor, an electrodermal activity sensor, a retina scan sensor, an eye movement tracking sensor, a pupil dilation sensor, a breathing rate sensor, a breathing depth sensor, and the like. It will be appreciated that a single sensor may detect more than one type of biometric parameter. The biometric parameter sensors may be included as part of a therapeutic device, and/or connected via wires or wireless link to a control module of the therapeutic device. In an aspect, some, or all, of the biometric parameter sensor information values may be obtained from sensors that are not part of the therapeutic device and/or are not coupled to the control module thereof. For example, a smart phone, or a smart watch, may detect some, or all, of the biometric parameters that are the basis for values provided as inputs to a learning algorithm of a therapy program. Thus, a smart phone or smart watch may generate biometric parameter information of a user during a therapy session, including a training period thereof, that may then be used as the basis for values that are input to a therapy program.

The system may also include a display interface that is in communication with the control module. The display interface may be a display screen attached to the therapeutic device/massage char that is easily viewable by a user using the therapeutic device. Or, the display may be part of a user's smart phone, or the display may be part of a user's other type of computing device, such as a PC, tablet, desktop computer, and the like.

The system may also include a processor in communication with the control module, which as described above may be part of the control module or may be located remote from the control module, to receive a selection of a first predetermined therapeutic program from among a plurality of therapeutic, or therapy, programs (different programs may focus on different areas of a user's body to provide therapy to), wherein the first predetermined therapeutic program includes one or more default therapeutic parameter values that correspond to operation of the one or more therapy-delivering components, and wherein the processor is further to modify one or more of the default therapeutic parameter values into at least one modified therapeutic parameter values based on signals produced by the one or more biometric parameter sensors that are interfaced with a particular user during a therapy session that the particular user uses the therapeutic device such that the one or more modified therapeutic parameter values differs from a corresponding one or more of the default therapeutic parameter values based on evaluation of at least one of one or more biometric parameter values derived from one or more signals from at least one of the one or more biometric parameter sensors during the therapy session.

In an aspect, the display interface and the processor are part of a user device that is not attached to the therapeutic device and wherein the user device and the control module of the therapeutic device communicate via a wireless link. In another aspect, the display interface and the processor are part of the therapeutic device.

In an aspect, the therapeutic device is a massage chair.

In an aspect, the processor of the system automatically selects the first predetermined therapeutic program based on a unique identifier of the particular user. The unique identifier may be based on a value communicated from the user's smartphone device via a wireless link, such as via a short rage link such as Bluetooth, or Wi-Fi. Or, the wireless link may be a long-range wireless link such as a cellular data wireless link as provide by a mobile network operator such as AT&T or Verizon. The unique identifier may be a unique identifier that the corresponding user chooses, such as a user name/pass work combination. The unique identifier of the user may be based on a unique identifier of the user's user device, such as a MAC address or an IMSI or a portion thereof, that is unique to the user's device, of a user's smartphone, smartwatch, or other wearable, or a friendly name of the user's smart phone, smart watch, or other wearable. The unique identifier of the user may be generated based on a biometric sensor such as a fingerprint scanner, a retinal scanner, a voice print recognition scanner, a hand geometry scanner, and the like. The unique identifier of the user may be a secret authentication code known to the user and to the processor.

In an aspect, the processor modifies the one or more of the default therapeutic parameter values into modified therapeutic parameter values based on signals generated by the one or more biometric parameter sensors and received by the processor during a predetermined parameter training period of a therapy session. The default parameters may be values used to initialize an artificial intelligence learning algorithm. The algorithm may include one or more of a variety of types of learning algorithms, such as supervised, unsupervised, or semi-supervised (i.e., includes supervised and unsupervised learning algorithms) and may include linear regression or other mathematical algorithms. In an aspect, the learning algorithm may not include a supervised algorithm. In an aspect, the learning algorithm may not include an unsupervised algorithm. During a training portion, or training period, of a therapy session, values corresponding to signals generated by the sensors may be processed by a DSP as inputs to the artificial intelligence learning algorithm. Other inputs to the learning algorithm may be therapeutic parameters values that control operation of the one or more therapy delivering components. The therapeutic parameter values are also typically outputs of the learning algorithm. In other words, known parameters that control operation of the therapy-delivering components, or their corresponding actuators, are processed by one or more DSP in conjunction with sensor signal values that reflect a user's actual physical response, or responses, to the operation of the therapy delivering components during training portion of a therapy session. The processing may compare the therapeutic parameter values and the sensors' signal values to predetermined desirable responses to the delivering of therapy to the user by the therapy-delivering components according to the learning algorithm. The predetermine desirable responses may be a range of values that have been normalized to values that result from the sensors' signals, or the sensor signal values many be normalized to comport with, accord with, agree with, or harmonize with, the range of values that are assigned in the learning algorithm to correspond to a given sensor's signal values. Thus, although the therapeutic parameter values may be outputs that are used to control operation of various therapy-delivering components, the therapeutic parameter values may also be inputs to the learning algorithm. The predetermined criteria that the DSP running the learning algorithm may apply to the inputs may include a target value for a given biometric parameter, such as heart rate. Other predetermined criteria may include a trend. For example, instead of a discrete target heart rate value with a +/− tolerance, a predetermined criteria may be that a user receiving therapy during a training session has a decreasing heart rate (a minimum heart rate may be appropriate for a desirable heart rate trend or a desirable blood pressure trend). Thus, in comparing biometric parameter information to predetermined criteria, the DSP may determine that for a given biometric parameter that a given sensor detects (i.e., heart rate, blood oxygen saturation percentage, body temperature, stress level, skin moisture level, blood pressure, electrocardiogram information, electromyogram information, electrodermal activity, a retina activity, eye movement, pupil dilation, breathing rate, breathing depth, and the like), the corresponding biometric parameter value is, or is not, within a predetermined range of a desired target value for the given parameter, or that the user's response during the training period is trending either in a desirable direction or in an undesirable direction. During the training period, the DSP may modify therapeutic parameter values to bring one or more given biometric parameter values within a tolerable range of a predetermined target value (i.e., the target value and the tolerance may together be considered criteria) or achieves a desirable trend and not an undesirable trend (i.e., the desirable trend may be considered a predetermined criteria). In an aspect, a trend criteria may be used during initial iterations of a learning algorithm, and then target value/tolerance criteria may be used during subsequent iterations to determine a final modified therapeutic parameter value during a training period.

In an aspect, the therapy session that includes the training period may be a first therapy session that the particular user has used the therapeutic device. For example, a purchaser of a massage chair may set up the chair, enter information that may be used to determine default therapeutic parameter values, and then the first time the user uses the massage chair with biometric parameter information signals associated with the user being provided to the control module the learning algorithm adjusts, updates, corrects, revises, or otherwise modifies the default therapeutic parameter values into modified therapeutic parameter values. If the DSP, or DSPs, determines during the session that the therapeutic parameter values have been optimized for the individual user, the DSP no longer continues to modify the therapeutic parameter values and the current modified therapeutic parameter values remain as the values that are used by the control module to determine control signals, or control values to send via signals, to one or more therapeutic component actuators.

In an aspect, the therapy session that includes the training period may not be a first therapy session that the particular user has used the therapeutic device. For example, during a previous training period the DSP, or DSPs, may have determined modified therapeutic parameter values that were then used to complete the remainder of a therapy session that a previous training period was a portion of. Or, previously modified therapeutic parameter values were used during a complete therapy session that did not include a training period. However, a user, or a user's doctor, may desire that a user further train a learning algorithm of a therapeutic program. Thus, upon making a selection via a user interface, a current therapy session may be programmed to include a new training period to further modify therapeutic parameter values of the therapeutic program. It may be desirable to further modify a therapeutic program's therapeutic parameter values for various medical reasons that may have altered the user's tolerance or response to therapy during a therapy session due to an illness, an injury, or passage of time and aging of the user since the last time therapeutic parameter values were modified. In addition, it may be desirable to modify therapeutic parameter values for different times of day, or for use following different activities, for example, and a different therapy program may be beneficial after an intense weight-lifting workout in the afternoon as compared to a program that may be more relaxing and more appropriate before bedtime after a long day of driving an automobile.

In an aspect, modified therapeutic parameter values may be stored remotely from the therapeutic device, such in a memory of a user's smart phone, a remote computer server such as maintained by a health care provider, or even downloaded from a publicly accessible web site such as a social media platform. (The latter scenario may be more appropriate for use in downloading generic therapeutic parameter values for use as default therapeutic parameter values.)

In an aspect, a set of one or more default therapeutic parameter values of the first predetermined therapeutic program are selected by the processor, which may be a general processor that may be different from a DSP processor that may be used to process inputs to the learning algorithm, based on a unique identifier corresponding to the particular user and based at least in part on medical history information corresponding the particular user's unique identifier.

In an aspect, a set of modified therapeutic parameter values determined during a previous therapy session for the particular user may be used as default therapeutic parameter values for a current therapy session for the particular user, such that the modified therapeutic parameter values as modified during the previous therapy session are further modified into new modified therapeutic parameter values during the current therapy session.

In an aspect, the processor, which may be a general processor that may be different from a DSP processor that may be used to process inputs to the learning algorithm, modifies at least one of the therapeutic parameter values into a modified therapeutic parameter value based on applying a correction factor to at least one of the signals received from the one or more biometric parameter sensors.

In an aspect, the processor determines at least one correction factor to apply to the at least one of the signals received from the one or more biometric parameter sensors based one or more environment conditions, including: environmental noise level, environment temperature, barometric pressure, a number of individuals proximate the therapeutic device, time of day, or time of year. In this aspect, a user may provide inputs to queries regarding environment conditions that may be used by the control module to bias biometric sensor signal information according to current environment conditions such that results of a training period (i.e., generation of modified therapeutic parameter values or new modified therapeutic parameter values during a training period) may take into account the effect that the environment conditions, such as increased stress that the presence of many people proximate the user, may have on the user's physiological responses to a therapy program during a training period.

In an aspect, a method comprises launching a therapeutic program, based on a unique identifier being input to or received by the therapeutic program, that operates therapeutic components of a therapeutic device, wherein the therapeutic program includes one or more therapeutic parameter values that are associated with the unique identifier and that are used by the therapeutic program to control one or more of the therapeutic components, wherein the unique identifier corresponds to a particular user of the therapeutic device. The therapeutic parameter values may be coefficients, factors, or other values that are determined during execution of a learning algorithm and may be outputs thereof. The therapeutic parameter values may also be inputs to the learning algorithm for processing during a training period. The therapeutic parameter values, or outputs of the learning algorithm, that are determined during a training period may be values that the control module of a therapeutic device, such as, for example, a massage chair, provides to individual therapy delivering components such as massagers of a massage chair.

The method may receive one or more current biometric parameter values generated during a therapy session that the therapeutic device provides therapy to the particular user, wherein the one or more biometric parameter values are derived from corresponding one or more biometric parameter sensor signals that are generated during the therapy session. The biometric parameter sensor signals, or biometric parameter values based thereon that are representative thereof, may be provided as inputs to the learning algorithm and may be used to modify the therapeutic parameter values used by the learning algorithm as outputs, which are used by a control application portion of a therapeutic program to control therapy-delivering components of the therapeutic device. The learning algorithm may modify the therapeutic parameter values in response to the biometric parameter values based on whether, or how well, the biometric parameter values match, correlate with, come close to, fall within, or otherwise satisfying biometric parameter criteria.

The method compares the one or more biometric parameter values to predetermined criteria, which may be biometric parameter criteria that corresponds to the biometric parameter values. For example, the target heart rate during a therapy session, such as massage session, may be 65 beats per minute (“bpm”). If during a training period, a user's heart rate is 80 bpm, the biometric parameter value for the heart rate parameter may not satisfy the criteria if the heart rate parameter has a tolerance of +/−5 bpm. Or, if during a training period of a therapy session a user's heart rate is 69 bpm during a first iteration of the learning algorithm but during a subsequent iteration of the learning algorithm the heart rate has risen to 70 beats per minute, even though the target heart rate criteria may be satisfied because number of beats per minute is within +/−5 bpm of the target heart rate of 65 bpm, the trend as a result of delivering therapy during a training portion of the training period of a therapy session has caused an increase in heart rate as evidenced by the movement away from the target heart rate. Thus, even though the target heart rate criteria may have been satisfied, the heart rate trend criteria may not be satisfied and therefore more iterations of the learning algorithm may be required to continue to refine therapeutic parameter values that are used to control therapy delivering components of the therapy device.

The method may, in response to the comparing of the one or more biometric parameter values to the predetermined criteria, which comparing may include evaluating current received biometric parameter values with a learning algorithm with the most recently acquired biometric parameter values as inputs to the learning algorithm, modifying the one or more therapeutic parameter values into modified therapeutic parameter values during the therapy session until current biometric parameter values satisfy the predetermined criteria.

When biometric parameter values satisfy corresponding predetermined biometric parameter criteria, such as heart rate or heart rate trend, the training period may be concluded and the method may continue to operate the therapeutic device according to the modified therapeutic parameter values as modified during the training period, or during a training portion of a training session. It will be appreciated that biometric parameter values are derived from signals from biometric parameters sensors that substantially continually generate new biometric parameter signals which in turn result in new biometric parameter values being derived during a training session or during a therapy session that is not a training period. Depending upon circuitry of the control module, or circuitry that interfaces with the biometric parameter sensors, a given sample rate may apply, for example, five samples per second. A most recent sample, or corresponding biometric parameter value, from a given biometric parameter sensor may be referred to herein as a current biometric parameter value for use with a current iteration of a learning algorithm to distinguish from a prior, or previously acquired, biometric parameter value that may have been used with a prior, or previous, iteration of the learning algorithm.

In an aspect, the comparing of the one or more biometric parameter values to predetermined criteria and the modifying the one or more therapeutic parameter values into modified therapeutic parameter values may be iteratively performed during a training period that ends when the biometric parameter values satisfy the predetermined criteria. More than one iteration of a learning algorithm may be performed before the modified therapeutic parameter values cause therapeutic components to deliver therapy, such as a massage, to the user such that biometric parameter values corresponding to the user's response to the therapy satisfy predetermined criteria.

In an aspect, the steps of the method may be performed by a processor of a device that is in wireless communication with the therapeutic device. For example, the steps of the method may be performed by a software application running on a user's smart phone that is in wireless communication with a processor of the therapeutic device, such as a processor of a control module of a massage chair. The wireless communication may be via a long-range wireless link, such as an LTE wireless link. Or, the wireless communication may be via a short-range wireless link such, as for examples, Wi-Fi or Bluetooth, or similar.

In an aspect, the sensors that generate the biometric parameter sensor signals may be part of a device that is in wireless communication with the therapeutic device, such as for example, a user's smart phone, or a smart watch. The smart watch may be in wireless communication with a user's smart phone, which may relay the sensor information to the therapeutic device via a wireless link. Or, the smart watch may be in wireless communication with a user's smart phone, which may process the sensor information and modifies therapeutic parameter values as described above before forwarding the therapeutic parameter values to the therapeutic device for use in controlling therapeutic components of the therapeutic device.

In an aspect, a massage chair comprises one or more therapy-delivering components operated by one or more corresponding therapy-delivering component actuators. A control module is communicatively coupled with the one or more therapy-delivering component actuators and is communicatively coupled with one or more biometric parameter sensors. The communicative coupling of the control module with the biometric parameter sensors may be via a wireless link to a user's smartphone, which obtains biometric parameter information, or derives biometric parameter values based thereon, from biometric parameter sensors. The massage chair may also comprise a processor in communication with the control module to receive a selection of a first predetermined therapeutic program from a plurality of predetermined therapeutic programs. Or, the processor may be part of another device besides the massage chair. Each of the plurality of therapeutic programs may correspond to a particular area of the individual user's body for which therapy is desired or may correspond to one of a plurality of different types of massage chairs or massage component types. The selection may be made via a user interface, such as a touch screen, hardware buttons, soft buttons, software control items, and the like. The selection may be made via an interface of a therapeutic program running on a user's smart phone device that is in wireless communication with the processor of the mass age chair. The first predetermined therapeutic program may include one or more default therapeutic parameter values that correspond to operation of one or more therapy-delivering components, which may include massagers, heating elements, cooling elements, vibration elements, and the like. The processor of the massage chair may also modify one or more of the default therapeutic parameter values into modified therapeutic parameter values based on biometric parameter values that are derived from biometric parameter signals received from one or more biometric parameter sensors that are interfaced with a particular user during a therapy session such that at least one of the therapeutic parameter values is modified during a training period based on at least one of the biometric parameter values received during the training period. The training period may be a stand-alone period or may be part of a complete massage session. If part of a complete massage session, the training period would typically occur during the beginning of the therapy/massage session.

### BRIEF DESCRIPTION OF DRAWINGS

FIG. 1 illustrates an environment of a therapeutic chair device that can receive control information from a user equipment device that can change operation of the therapeutic chair.

FIG. 2 illustrates a flow diagram of a method for modifying therapeutic parameter values used to control operation of a therapeutic device based on biometric sensor feedback during a therapy session for a particular therapy session user.

FIG. 3 illustrates a user equipment smart phone device running an application that provides a user interface for interfacing with a massage chair therapeutic device.

FIG. 4 illustrates a user equipment tablet device running an application that provides a user interface for interfacing with a massage chair therapeutic device.

FIG. 5 illustrates a therapeutic device/massage chair having a finger sensor for obtaining biometric parameter information and providing same in a signal that is used by a program that operates/controls the therapeutic device during a therapy session.

FIG. 6 illustrates a diagram of a neural network aspect of a therapeutic program that may be trained during a training period to derive therapeutic parameter values that are outputs of a learning algorithm depicted by the neural network.

FIG. 7 illustrates a roller massager, that may be a stand-alone therapeutic device as described herein, or that may be a therapeutic component of a massage chair therapeutic device; the massage rollers are shown providing therapy to targeted areas of an individual user receiving a therapy session.

FIG. 8 illustrates airbag massagers that may be therapeutic components of a massage chair therapeutic device.

### DETAILED DESCRIPTION

As a preliminary matter, it will be readily understood by those persons skilled in the art that the present invention is susceptible of broad utility and application. Many methods, embodiments, and adaptations of the present invention other than those herein described as well as many variations, modifications and equivalent arrangements, will be apparent from or reasonably suggested by the substance or scope of the present invention.

Accordingly, while the present invention has been described herein in detail in relation to preferred embodiments, it is to be understood that this disclosure is only illustrative and exemplary of the present invention and is made merely for the purposes of providing a full and enabling disclosure of the invention. The following disclosure is not intended nor is to be construed to limit the present invention or otherwise exclude any such other embodiments, adaptations, variations, modifications and equivalent arrangements, the present invention being limited only by the claims appended hereto and the equivalents thereof.

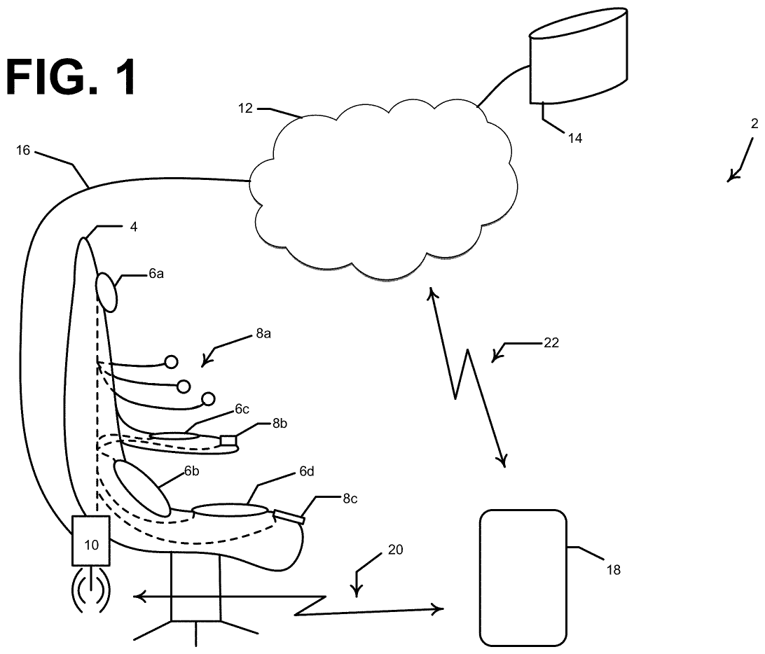

Turning now to the figures, FIG. 1 illustrates an environment 2 showing a massage chair 4. A user may use the massage chair 4 four therapeutic purposes. The massage chair may include therapy-delivering components 6a-6d that are operated by therapy delivering component actuators (not shown in the figure). The massage chair may also include biometric parameter sensors 8a-8c. The therapy-delivering components 6a-6d and the biometric parameter sensors 8a-8c may be coupled to control module 10, which may include a processor. (Control signaling connections of therapy-delivering components and biometric parameter sensors are shown in dashed lines; power supply connections are not shown for clarity.) Control processor 10 may be coupled to communication network 12 which may be in communication with data store 14. Massage chair 4 may communicate with communication network 12 via communication link 16, which may be a wired link, or a wireless link such as for example an LTE cellular link or similar. Control module 10 may communicate with a user device 18 via communication link 20, which may be a wired or wireless link, and the user device may communicate with communication network 12 via a wired or wireless communication link 22 such as an LTE cellular link or similar. A user may undergo a therapy session in massage chair 4 by sitting in the chair and attaching at least one of sensors 8a-8c to his, or her, body. The user may select a therapy program with a display or user interface which may include a display screen that is part of chair 4. Preferably, the user undergoing a therapy session using chair 4 selects the therapy program with an application running on user device 18. The application running on user device 18 may access a unique identifier corresponding to the user from within the user device and may provide the unique identifier of the user to control module 10 of chair 4 via link 20, which may be a Wi-Fi or Bluetooth short range wireless link. Based on the unique identifier provided by the application running on user device 18, control module 10 may retrieve a set default therapeutic parameter values that have been previously stored therein that correspond to the unique identifier. Or, the application running on a user device 18 may provide a set of default therapy parameter values to control module 10 for use in a current therapy session where the user uses therapeutic device chair 4 for therapy.

If a current therapy session that a user of chair 4 is about to undergo, or is undergoing, is an initial use of the massage chair, the set of default therapy parameter values may be selected based on user-descriptive inputs via the application running on user device 18. User-descriptive inputs, may include, for example, gender, age, height, weight, and any medical conditions associated with the user that are selected from a set of predetermined medical conditions, etc.

As shown in the figure, therapy-delivering component 6a and its associated therapy-delivering component actuator may compose an upper back or neck massager. Therapy-delivering component 6b maybe a lower back massager. (Therapy-delivering components may be referred to herein as therapeutic components.) Therapy-delivering components 6c and 6d may be an arm massager and a leg massager, respectively. Biometric sensors 8a may be sensors that attach with an adhesive to a user's chest, back, or abdomen. Biometric sensors represented by sensors 8a may also include a blood pressure cuff that is monitored by and controlled by control module 10. Biometric parameter sensor 8b may be a sensor that surrounds, touches, grips, pierces, or otherwise interfaces with one of a user's fingers. Such a sensor as sensor 8b may monitor a user's pulse rate, a user's blood oxygen level, a user's blood sugar level, a user's blood pressure, a user's body temperature, a user's skin moisture level, and the like. Biometric parameter sensor 8c may be a leg sensor that may monitor muscle tension, muscle movement, or other biometric parameters that may be indicated by changes in pressure, temperature, moisture, tautness of a muscle, in a user's leg.

In an aspect, a user in possession of user device 18 may, in proximity of therapeutic device 4, use an application, or ‘app,’ running on the user equipment device, which may be a smart phone, and transmit authentication information, such as a user name and password, which may be encrypted, to control module 10. Control module 10 may configure operation of a therapy session based on the therapeutic parameter values associated with the user authentication credentials received from user equipment device 18. For example, a data store in, or communicatively coupled with, control module 10 may associate parameter values that control speed, intensity, movement displacement, heating, cooling, or other operations of components 6 with a particular user. Perhaps a user tends to have upper back and neck tightness, but does not have any malady associated with his, or her, legs. Upon receiving authentication information that authenticates the user's user equipment device 18 with control module 10, and thus with therapeutic device 4, the control module may receive an input from a pressure sensor of chair 4 indicating that the user has sat down on, or laid down on, the chair to begin a therapy session. Or, control module 10 may receive an indication from one or more of sensors 8 that the sensors have been placed on, connected to, or attached to the user. Upon authenticating a user, or retrieving from a data store therapeutic parameter values that are associated with a user, the control module may cause therapy-delivering components to begin operation according to the therapeutic parameter values when the user pushes a ‘start’ button or control, that may be part of therapeutic device 4 or may be part of the application running on user device 18. In the scenario described above where the user tends to have upper back stiffness or neck stiffness, the parameter values associated with component 6a may cause control module 10 to send control signals, or messages, (which may be analog or digital) to the actuator associated with component 6a. The therapeutic parameter values may cause component 6a to gently massage the areas of the user's body that are touching the component, namely the user's upper back and neck regions. At the same time, since the particular user does not tend to have leg tension, therapeutic parameter values may cause component 6d to remain motionless. It will be appreciated that a given therapeutic component may be capable of performing multiple functions in response to signals or messages from control module 10 that are based on therapeutic parameter values that correspond to the given therapeutic component. For example, even though a user may only need a neck massage and no leg massage, component 6d may produce warmth for the user in the leg area even though the massage functionality is not currently implemented because a massage parameter value for component 6d may be 0 (on a scale of 0-10) but a warming parameter value that operates a warmer of component 6d may be 2 on a scale of 0-10.

In an aspect, control module 10 may determine that the heart rate of a particular user during a therapeutic session may have risen in lockstep with the massage action of component 6a beginning. If a therapeutic parameter value for component 6a is 8 as a default value, or as a beginning value, at the beginning of a therapy session, control module 10 may determine that the therapeutic parameter massage value of 8 that controls operation signals sent from the control module to the massage component should be modified to a reduced value of 4, for example, in case the massage action performed by component 6a is too aggressive and caused the elevated blood pressure.

In an aspect, instead of control module 10 performing the modification to therapeutic parameter values of a therapy program that control the components of chair 4, a user's smart phone 18 may receive (typically wirelessly) session information from sensors 8 and may determine that the massage parameter value that controls component 6a should be modified from a default value of 8 to a reduced value of 4 based on sensor information from one of sensors 8a that the user's hear rate and blood pressure have risen during operation of component 6a according to a massage parameter of 8. If the modified therapeutic parameter value that has been reduced results in a lower heart rate and blood pressure of a particular user during a session than at the default value, the modified therapeutic parameter value may be stored as modified therapeutic parameter value, or as a new modified therapeutic parameter value if the default parameter value for the current session was a modified parameter value that resulted from modification during a previous therapeutic session with the particular user using chair 4.

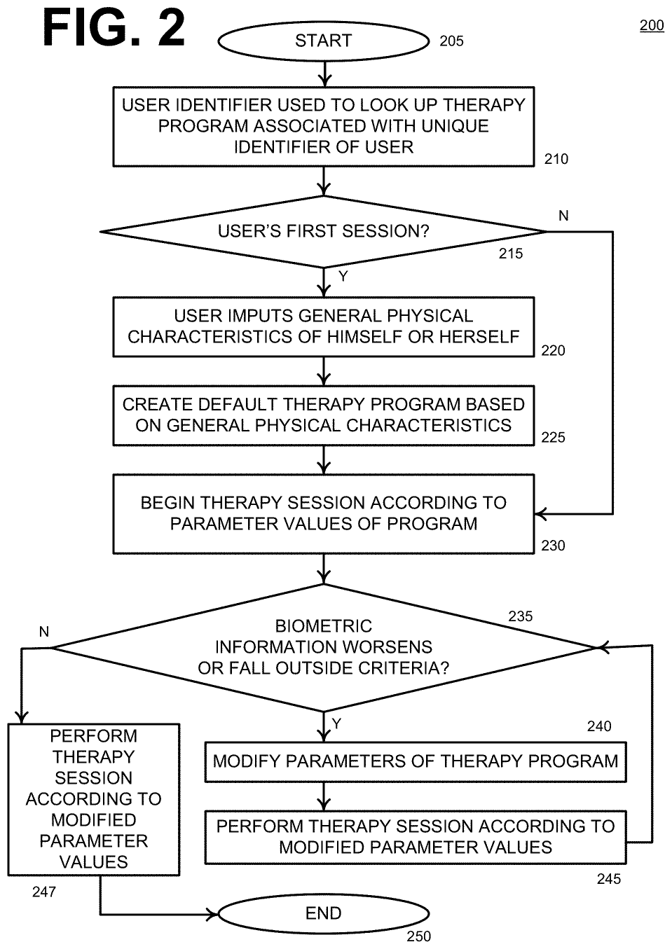

Turning now to FIG. 2, the figure illustrates a flow diagram of a method 200 for using a therapeutic device to improve one or more various medical or physical conditions. Method 200 begins at step 205. At step 210 a unique user identifier is provided to look up a therapy program that is associated with the user identifier. The unique user identifier may be provided by a user who intends to use the therapeutic device via a user interface of the therapeutic device. Or, the user identifier may be provided via a user interface of a user equipment device such as, for example, a smart phone. The user identifier may be stored within the user equipment device and stored via an application running on the user device. The user device may provide user identifier or authentication credentials that the therapeutic device may use in determining the user identifier.

The therapeutic device may include a control module that stores therapy session programs, or therapeutic session programs, which program or programs may include therapeutic parameter values. The therapeutic parameter values may be used to determine or provide control information to therapeutic components of the therapeutic device, which therapeutic components may provide massage, heating, cooling, vibration, or other stimulation to a user of the therapeutic device. A user interface of the therapeutic device may be used by a user to select a therapy program from among a plurality of therapy programs. Typically, differences among therapy programs result from differences in therapeutic parameter values of a given program. The therapeutic parameter values may be coefficients, factors, or other types of values that may be used to generate a signal based on the values to cause one or more of the therapeutic components to operate according to the signal and the therapeutic parameter value that it is based upon.

In an aspect, a user's smart phone may launch, or run, an application that can access therapeutic parameter values for a given therapeutic session program and that can provide therapeutic parameter values for a selected therapeutic session program to a control module of the therapeutic device. Thus, a user may select a therapeutic session program via a user interface of the therapeutic device, or a user may select the therapeutic session program using an application on a device that is in communication with the therapeutic device, wherein such devices that are in communication with the therapeutic device may include a smartphone, a laptop, a tablet, a desktop computer, a telephone, or other type of communication device.

Continuing with discussion of method 200 illustrated in FIG. 2, at step 215 a determination may be made whether a therapeutic session to be performed by the therapeutic device is a first therapeutic session of a particular user. If the therapeutic session to be performed by the therapeutic device is not a first therapeutic session for the particular user, method 200 advances to step 230.

If the therapeutic session to be performed by the therapeutic device is a first therapeutic session for the particular user, method 200 advances to step 220. At step 220 a user may provide general physical characteristic information of himself, or herself. Such general physical characteristic information may include, for example, age, height, weight, gender, known medical conditions, recent emotional situations that involved the user, or any other information that might be relevant to a user's current physical state. The user may provide general physical characteristic information via a user interface of the therapeutic device, via an application or user interface of the user's user device, or via any other user interface or device that provides the user interface and that is in communication with the therapeutic device (i.e., in communication with a control module of the therapeutic device).

At step 225 a default therapy session program is created for the user associated with the unique identifier discussed in reference to step 210 based on the general physical characteristic information that was provided at step 220. The default therapy session program may be created by the control module of the therapeutic device. The default therapy session program may be created by an application running on a user's smart phone or other device that is in communication with the therapeutic device's control module, a device that can provide stored data to the control module via a disk, card, flash drive, or other similar means of storing data or information, or by any other device such as a desktop computer that is communicatively coupled with the therapeutic device's control module. The default therapy session program may be customized to the particular user to cause certain therapeutic components to operate according to corresponding therapeutic parameter values, which may be outputs, or output coefficients of variables, of a learning algorithm. Accordingly, the default therapeutic parameter values may cause a leg massager to operate with a certain intensity (i.e., extent of massaging motion), a certain frequency (i.e., speed of the massaging motion), and for a certain duration for the particular user based on the general physical characteristic information provided at step 220.

At step 230, a user begins a therapy session to be produced by the therapeutic device. Before the therapeutic device delivers, or produces, therapy the user may attach to, place on, insert, pierce, or otherwise interface one or more biometric parameter measuring sensors to his, or her, body. These sensors may measure such biometric parameters as heart sound, heart/pulse rate, body temperature, blood pressure, electrical signaling of the heart, brain waves, eye motion, skin temperature, breathing rate, air intake during breathing, skin moisture, blood oxygen level, or other bodily/physiological changes that may be measured in a nondestructive and relatively noninvasive manner. The sensors may be attached via one or more conductors to a control unit of the therapeutic device, or may be part of a user's device, such as a smart phone or a smart watch or other wearable device. After attaching, touching, or otherwise interfacing with one or more biometric parameter measuring sensors a user may cause a therapeutic device to begin delivery of a therapy session by a variety of actions, including pressing a button, selecting an icon via user interface, providing a verbal command to start, squeezing a sensor that may or may not be one of the biometric parameter measuring sensors, or by other similar interface which may include haptic interfaces. A therapy session program may also be started by a user remote from the therapeutic device, such as a doctor, nurse, or other individual remotely monitoring the therapy session via a computer device that is in communication with the control module of the therapeutic device. In an aspect, a therapy session may be started automatically, by operation of a timer, or upon the occurrence of another triggering device, such as another user using another therapeutic device substantially simultaneously with the particular user of the therapy session that begins at step 230.

After a therapy session has begun at step 230, information contained in biometric parameter sensor signals from the biometric parameter measuring sensors are monitored during the therapy session at step 235. The monitoring may be performed by a control module of the therapeutic device, or signals generated by the parameter measuring sensors may be transmitted to one or more devices remote from the therapeutic device, such as a user's smartphone, smart watch or other wearable, tablet, laptop, remote internet-connected computer for monitoring by an application running on such one or more devices. Regardless of whether the control module of the therapeutic device monitors information contained in signals generated by the parameter measuring sensors, or whether an application running on a user's smart phone or smart watch, for example, monitors information contained in signals generated by the parameter measuring sensors, a determination is made at step 235 whether the therapy session delivered by the therapeutic device has caused one or more of the biometric parameters measured by the sensors to worsen relative to predetermined criteria (for example where the criteria is trend criteria), or whether the biometric parameter information falls outside of predetermined criteria (for example where the criteria is a target value with a tolerance). The predetermined criteria may be ranges of values that correspond to the one or more biometric parameters and an alert may be provided, for example visually or aurally, to a user that a given monitored biometric parameter does not satisfy the criteria.

Preferably, the predetermined criteria may be applied by a learning algorithm such that outputs of the learning algorithm, which outputs may be therapeutic parameter values, are modified during a training period of a therapy session so that the therapeutic parameter values control operation of corresponding therapeutic components of the therapeutic device providing the therapy session to bring the biometric parameters within the predetermined criteria, or at least closer to satisfying the criteria than when the therapy session began.

If operation of the therapeutic device in delivering therapy to a user during a therapy session that began at step 230 has resulted in a determination at step 235 that the therapy session has caused biometric parameter information of the user to worsen or that the biometric parameter information exceeds predetermined criteria, method 200 advances to step 240. At step 240 the control module of the therapeutic device, or the application running on the user's smart phone that may be monitoring information in signals generated by the sensors, may modify therapeutic parameter values of the therapy session program that were either generated as part of a default program at step 225, that were generated during a previous iteration of step 240 during the present execution of method 200, or that were previously generated during a previous therapy session and that were used at step 230 if, for example, the current therapy session was determined not to be the particular user's first session at step 215.

As an example, if a default program generated at step 225 includes a therapeutic parameter value that causes a back massage therapeutic component of the therapeutic device to deliver an aggressive back massage, (e.g., a motor that creates massaging motion of the massaging component on the back of a massage chair causes massaging displacement of one inch at a frequency of thirty cycles per second) and a biometric parameter sensor interfaced with the user who is undergoing the therapy session indicates at step 235 that the user's blood pressure, heart rate, or skin moisture levels are increasing during the therapy session, the control module of the therapy device, or the application running on the user's smart phone that is monitoring sensors signals from the biometric parameter sensors, may determine that a massage motion having a displacement of half an inch at a frequency of twenty cycles per second would be more appropriate to try for the current therapy session for the particular user undergoing the current therapy session. Accordingly, the control module of the therapy device, or an application running on the user's smart phone, may modify a therapeutic parameter value, or therapeutic parameter values, of the therapy session program that is used to generate a control signal to the back massaging component of the therapeutic device such that the massaging action displacement and massaging frequency are reduced in accordance with the modified therapeutic parameter value, or values. Thus, the default therapy session program may be modified to include modified therapeutic parameter values, or a previously modified therapy session program may be modified into a new modified therapy session program that includes the modified therapeutic parameter values that were determined at step 240.

At step 245 the current therapy session that the particular user is currently undergoing may continue according to therapeutic parameter values that are included in a newly modified therapy session program wherein the therapeutic parameter values of the newly modified therapy session program were modified at step 240 in response to a comparison of biometric parameter sensor information to predetermined criteria at step 235. Method 200 returns to step 235 where monitoring of sensor information continues as discussed above. If the modified therapy session program therapeutic parameter values have not resulted in bringing biometric parameter information contained in signals generated by the biometric parameter sensors within predetermined criteria, or if the biometric parameter information represented in the signals worsen based on comparison to the predetermined therapeutic criteria, steps 240 and 245 may be repeated. Steps 235-240 may be referred to as a training period of a therapy session when the therapeutic parameter values are evaluated and modified as outputs (and in some cases as inputs too) of a learning algorithm.

If the modified therapeutic parameter values of the newly modified therapy session program have brought biometric parameter information contained in one or more signals generated by the biometric parameter sensors within a predetermined criteria, or if at least the monitored biometric parameter information contained in the signals shows improvement in the biometric parameters as compared to the predetermined criteria as a result of the newly modified therapy session program therapeutic parameter values, method 200 advances to step 247 and the therapeutic device continues providing therapy during the therapy session until a predetermined therapy session duration, which may be a therapeutic parameter value, elapses and method 200 ends at step 250.

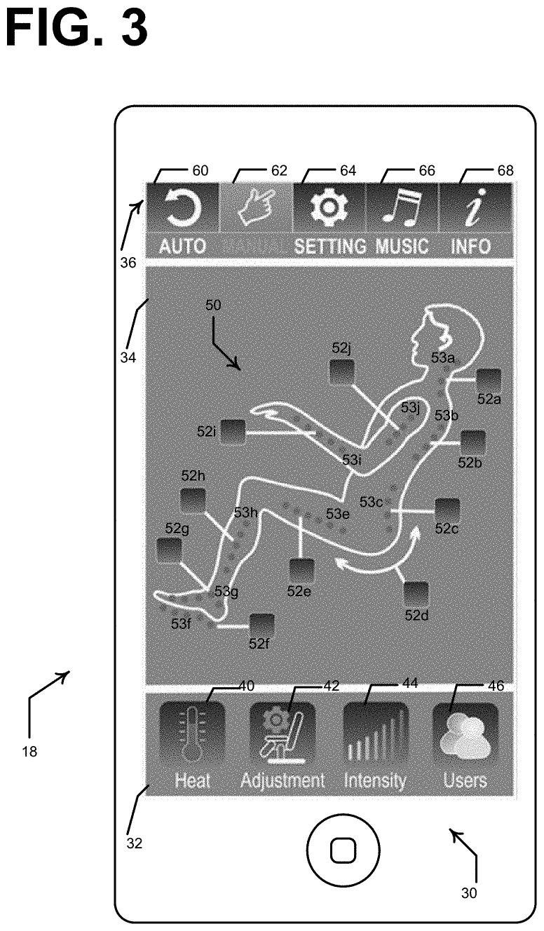

Turning now to FIG. 3, the figure illustrates a smart phone 18 running an application 30. Application 30 includes a therapeutic parameter control portion 32, a therapeutic component interactive display portion 34, and a general features selection portion 36. Therapeutic parameter control portion 32 shows a temperature selection icon 40, a therapeutic component adjustment icon 42, a therapeutic component intensity icon 44, and a user selection icon 46. Therapeutic component interactive display portion 34 includes a diagram 50 of a user's body sitting in a therapeutic device, which as shown in the figure may be a massage chair. Therapeutic component interactive display portion 34 includes therapeutic component selection items 52a-52j, which correspond to therapeutic component icons 53a-53j, which generally represent therapeutic components 6 shown in FIG. 1. General features selection portion 36 in FIG. 3 includes a program Auto mode selection item 60, a manual selection item 62, an application settings selection item 64, a music selection item 66, and an information or help selection item 68.

Before a user begins use of a therapeutic device for a therapy session the user using application 30 may use user selection item 46, which may be a drop-down box, a list of users, a text box for entering a user (and perhaps password), and the like. For example, if the user selection item 46 is a drop down box displaying multiple users who may frequently make use of application 30 in controlling a therapeutic device, the user selection item may provide a dynamic link or reference such that when a user selects his name from the drop down box application 30 causes smartphone 18 to transmit to a control module of a therapeutic device a unique identifier of the user selected using user selection item 46. The unique identifier of the user may be a common name, or another alphanumeric textual name that the user has previously selected as referring to him, or her. The unique identifier of the user transmitted by smartphone 18 to control module of the therapeutic device may be authentication credentials associated in a database within user device 18 such that therapeutic parameter values associated with a particular user corresponding to the authentication credential are applied to a program that controls a therapeutic device in providing therapy to the particular user during a therapy session.

In an aspect, upon a selection being made of a particular user using selection item 46 application 30 may retrieve previously stored therapeutic parameter values from within smartphone 18 and may communicate the retrieved therapeutic values corresponding to that particular user to a therapeutic device which may then use the received therapeutic parameter values to control one or more therapeutic components during a therapy session provided by the therapeutic device. As shown in FIG. 3, operation of the therapeutic device as indicated by operation mode item 60 is set to auto, or automatic. When in automatic mode, upon selecting a particular user with selection item 46 therapeutic parameter values are automatically applied at the control module of the therapeutic device regardless of whether smartphone 18 transmitted the therapeutic parameter values to the control module, whether the control module itself retrieved stored therapeutic parameter values based on a unique identifier received from the user device, or whether the parameter values were received from a user selection made directly on a user interface of the therapeutic device. The therapeutic device may retrieve therapeutic parameter values from a data store within itself or within its control module, or from a remote location such as, for example, an Internet connected server if the therapeutic device control module is communicatively coupled with the Internet.

A user may override therapeutic parameter values that have been associated with his, or her, unique identifier by selecting manual mode using mode selector item 62. When in manual mode, a user may select or highlight one or more therapeutic components 53a-53j by touching one or more of corresponding therapeutic component selection items 52a-52j. For each one or more component selection items 52a-52j selected a user may manually modify one or more corresponding therapeutic component parameter values using parameter selection items 40, 42, or 44. It will be appreciated that selection items 40, 42, and 44 are given for purposes of illustration and that other selection items corresponding to other therapeutic parameter values as discussed elsewhere herein may also be presented in parameter control portion 32.

In addition to providing a way to manually override parameter values for control of therapeutic components 53, therapeutic component selection items 52 may also provide parameter values assigned to corresponding therapeutic components 53. For example, instead of boxes, as shown in FIG. 3, representing component selection items 52, alphanumeric textual information may be displayed instead that provides parameter value information for application to the corresponding therapeutic component during a therapy session. The display of the alphanumerical textual information may include default parameter values for the given corresponding therapeutic component or may include modified therapeutic component values, which may be, or may have been, dynamically updated, revised, or modified during a therapy session as discussed above in reference to FIG. 2. Depending upon which parameter selection item 40, 42, or 44 is selected, or are selected, component selection items 52 may display as alphanumerical textual information one or more parameter values corresponding to the parameters that correspond to the one or more selection items that a user has selected using selection items 40, 42, or 44. In an aspect, Manual mode selected by item 60 may be used to initialize, or create, default therapeutic parameter values before a training period.

General application selection item 64 may provide a user with options relating to application 30, for example, background color, language, wireless connectivity mode, whether to provide therapeutic parameter values to others via a communication network in communication with smartphone 18, or other settings related to the interfaces of application 30. General application selection item 66 may provide a user a way to select music or other entertainment programming for his, or her, enjoyment during a therapy session.

Information selection item 68 may provide such information as application version, sharing history, access to previous therapeutic parameter values of a therapy program, notifications of updated features of application 30 that may be available, and the like. Information selection item 68 may also provide a way for a user to view current biometric parameter information during a therapy session or historical biometric parameter information monitored and recorded during one or more previous therapy session(s).

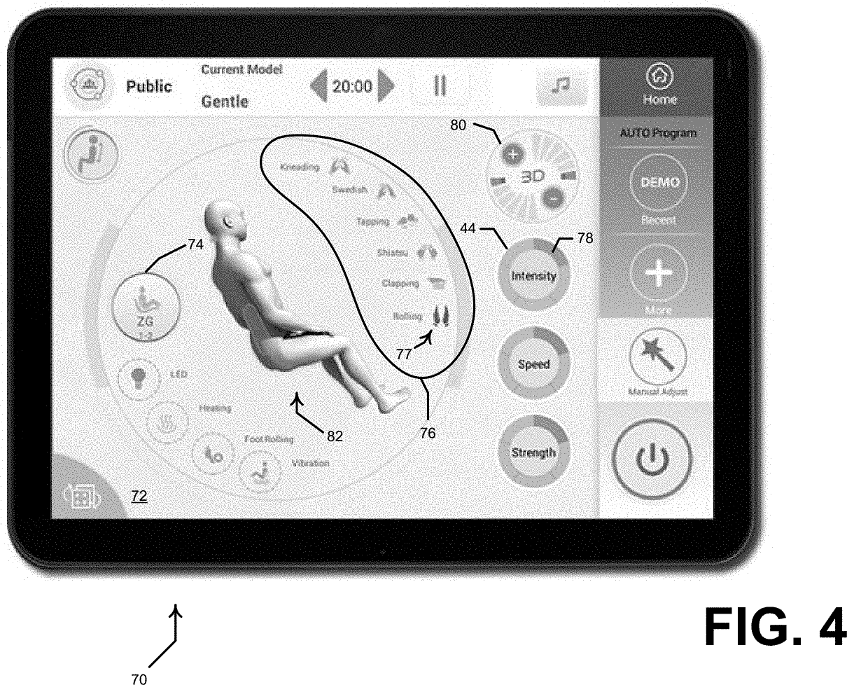

Turning now to FIG. 4, the figure illustrates a user equipment device tablet 70 running applications 72 that provides a user interface for interfacing with the controlling the therapeutic massage chair device. Application 72 is somewhat similar to application 13 shown in FIG. 3 and includes a parameter selection by and for intensity 44 is shown in FIG. 3. Continuing with discussion of FIG. 4, selection item 74 is indicated as being selected. Selection if item 74 selects massage components for further refinement of massage operation according to selected items 76. The massage type selection item 77 is shown as selected (because it is not shown dimmed as are the other massage type selection items) and shows that a user has selected a rolling type of massage. Other massage type selection items of the group 76 of massage type selection items include kneading, Swedish, tapping, Shiatsu, and clapping in addition to the rolling type massage selection item 77. The interface of applications 72 also shows that a user has adjusted intensity selection item 44 to an intensity level of about 20% based on the darker shaded annular portion 78 of the intensity selection item. A user enjoying a therapy session may manually increase or decrease the intensity of massage component during his, or her, therapy session by manually touching intensity selection item 44 with a finger and increasing or decreasing a shaded annular portion 78 of the intensity selection item. Or, manual selections may be used to generate default therapeutic parameter values. When a user has placed application 72 in automatic mode, feedback from biometric parameter sensors may be used to modify therapeutic parameter values, for example, a massage intensity during a training period, or if training is not required, to perform a massage according to already-determine therapeutic parameter values. Shaded annular portion 78 of intensity selection item 44 may automatically increase or decrease in size relative to the unshaded portion of the annular ring that surrounds the intensity selection item based on a determination made by application 72 that massage intensity is insufficient or is too intense, respectively. Application 72 also includes a view perspective selection item 80 whereby a user may manually scroll the perspective selection item to cause rotation and inclination changes of human figure 82 in correspondence therewith.

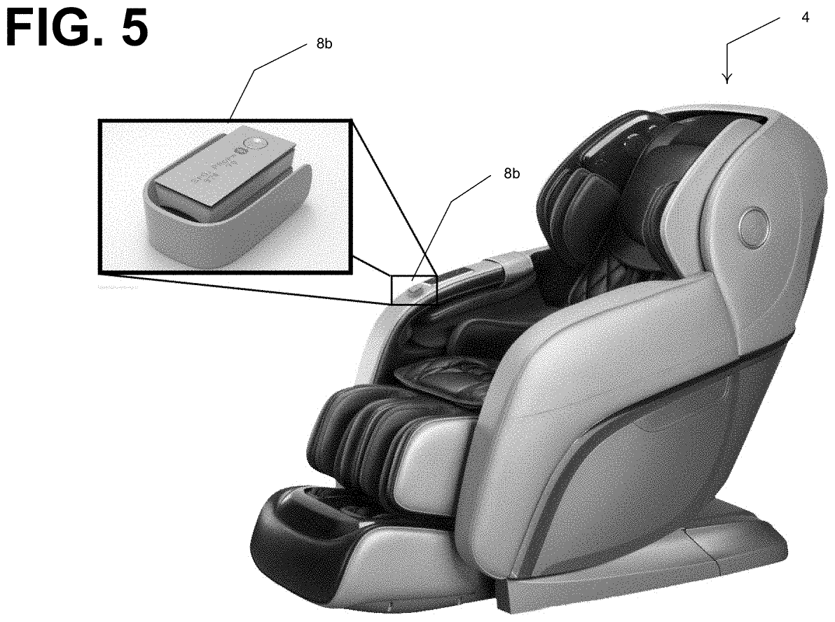

Turning now to FIG. 5, a therapeutic device 4 is shown as a massage chair. Massage chair 4 includes a finger sensor 8b as described above in reference to FIG. 1. Sensor 8b may provide biometric parameter information signals to a control module of device 4 which may determine that modifications to therapeutic parameter values should be adjusted in response to the biometric parameter information signals provided by the sensor. Or, the biometric parameter information signals from sensor 8b may be provided to a user device such as a smart phone or tablet either directly from the sensor or to a control module of therapeutic device four which may then forward information contained in the therapeutic parameter information signals to a user device, which may then determine that modifications to therapeutic parameter values should be made in response to information contained in the signals that were acquired during a therapy session from the sensor(s).

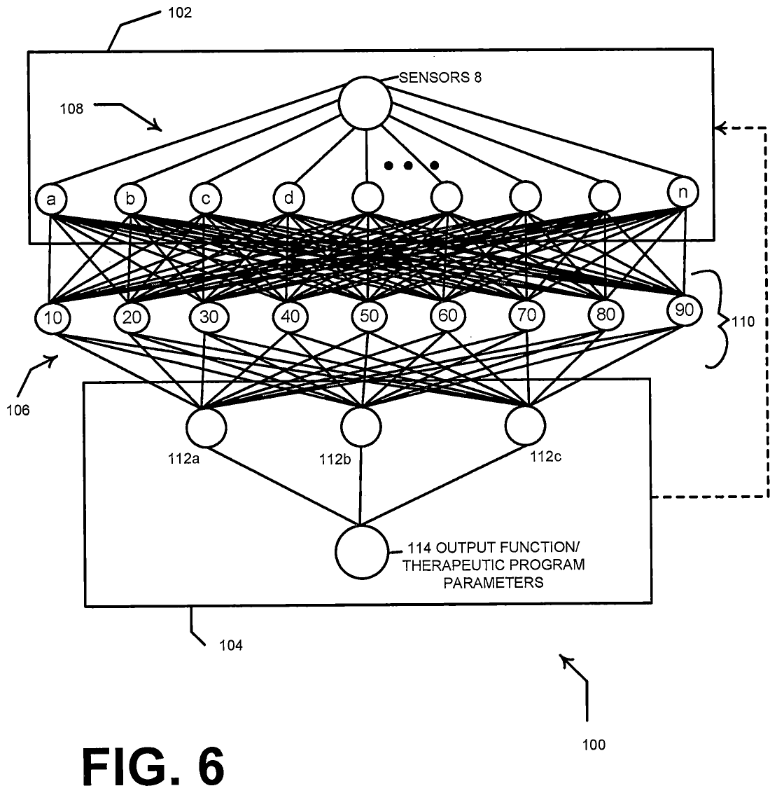

Turning now to FIG. 6, the figure shows a diagram of an example neural network aspect. The diagram illustrates neural network 100, which includes inputs 102, outputs 104, and nodes 106 of hidden layer 110. Inputs include biometric parameter input values 108a-n, which may be derived from signals from one or more sensors 8 as discussed above in reference to other figures. Input value 108a may represent heart rate measured by a heart rate sensor, input value 108b may represent blood oxygen percentage from a blood oxygen sensor, input value 108c may represent blood pressure from a blood pressure cuff, or similar sensor, and input value 108d may represent a stress level of a user using a therapeutic device during a therapy session that may be determined from a sensor that measures a user's skin moisture level. Other inputs to 108n may also be provided to hidden layer 110, and may include body temperature, electrocardiogram information, electromyogram information, electrodermal information, and the like. Outputs 104 may include a therapeutic parameter value 112a which may control operation of a heating element in a therapy component, a duration value 112b may control duration of a therapeutic component, and output value 112c may be a value that controls vibration intensity of a therapeutic component. Hidden layer 110 may include nodes 106 that may be used to determine output values 112 based on input values 108. For simplicity, network 100 may be viewed as determining outputs for a given therapeutic-delivering component, but the network could be expanded to show multiple sets of outputs to multiple corresponding therapy delivering components, which would be unwieldy to show in the figure but that could be illustrated with multiple layers of outputs, multiple hidden layers, and multiple input layers.

Hidden layer 110 may include a plurality of nodes corresponding to normalized biometric parameter input values. For example, if a range of default biometric parameter input values 108c corresponds to a systolic blood pressure value range of 81 to 170, an input value 108c may be normalized such that the corresponding normalized value may correspond to a value between 10 and 90 as shown in nodes 106 of hidden layer 110. For example, a systolic blood pressure value between 81-90 may correspond to a normalized value of 10, a systolic blood pressure value of 91-100 may correspond to a normalized value of 20, and so on. Processing at nodes 106 may modify predetermined, or default, weight factors, coefficients, variables, or other types of values that are used to determine therapeutic parameter values. If a biometric parameter input value for systolic blood pressure 108a corresponds to a normalize value of 20, a blood oxygen concentration level corresponds to a normalize value of 50, a heart rate input value 108a corresponds to a normalized value of 80, and a stress input value 108d corresponds to a normalized stress level of 90, hidden layer 110 may compute output values 112a, 112b, and 112c that are in the middle of ranges for corresponding therapeutic parameter values, and thus would cause operation of therapeutic components somewhere between minimum and maximum operation output (i.e., not a maximum or minimum heat output, not a maximum or minimum duration massage, and not a maximum or minimum massage vibration intensity). The output values 112a, 112b, 112c, 112d, . . . 112n, may be based on weight factors that are computed by network 100 during training of the network. Weight factors of the network may be based on whether an input value 108 falls within a range corresponding to a respective node 106 such that the closer an input value 108 is to 50, for example, a weight factor for a term of a function of network 100 may be close to 1.0 for the ‘50’ node, and close to 0 for the ‘40’ and ‘60’ nodes. However, the weight factors for ‘40’ and ‘60’ nodes may not be exactly 0 to indicate that for a given biometric parameter input 108 the value therefore is not exactly normalized to 50. The weight factors may be used by an output function of network 100 to calculate therapeutic parameter values that are used to control operation of therapeutic components of a therapeutic device during a therapy session. A therapy session, or a portion of a therapy session, may be a training session, or a training period, that is used to refine or modify therapeutic parameter values which correspond to output values 112.

In addition, trained output values may be used as default therapeutic parameter values during a current training period or during another training period to further refine the output values as a user's body reacts to revised outputs and corresponding therapeutic parameter values 112. A user's reaction to revise/modified output values 112 may be reflected in sensor signals that are the basis for inputs 108. Thus, a user of a therapeutic device for one or more therapy sessions may obtain a customized program for controlling therapeutic components of a therapeutic device, which program may include a trained set of output values that correspond to trained therapeutic parameter values based on his, or her, body's response to the operation of therapeutic components during a training session, or sessions. Further refinement is shown in the figure by the dashed line that leads from output section 104 back to input section 102. The dashed line from output section 104 to input section 102 is shown as a broken line to illustrate that feedback of output values 112 may be used as input values to hidden layer 110 for further refinement of output values during a training session may be performed. Such use of feedback of output values as inputs values may be useful to refine output values based on a desirable trend in biometric parameter values, for example, improvement of oxygen percentage in a user's blood as information in an input 108b signal trends toward 100% blood oxygen saturation. On the other hand, in another aspect feedback of output values as inputs is not used during a training period during a session. Such non-use of output values as input values may be chosen by a thereapeutic program if a modification of therapeutic parameter values is desired such that a given biometric parameter value, for example heart rate, falls within a predetermined target criteria, such as a range from 50-80 beats per minute (which beats per minute range should not to be confused with normalized values of heart rate, which may correspond to processing at normalized node value 40 of layer 106).

The paragraphs above describe methods, systems, and apparatuses for training a machine learning algorithm that is used to control therapeutic delivering components of a therapeutic device, such as a massage chair, in providing a therapy session to an individual who is identified to the therapeutic device, to the learning algorithm, or to a therapeutic program that launches, includes, manages, or can exchange information with, the learning algorithm via a unique identifier that corresponds only to the particular individual. During a training period which may be a stand-alone therapy session, or a portion of a therapy session provided to a user by a therapeutic device, biometric parameter values that are derived from signals generated by biometric sensors that are attached to, or otherwise interfaced with, the particular user, are used by the learning algorithm to refine, change, or modify therapeutic parameter values that are used to control therapy-delivering components of the therapeutic device that is providing therapy to the individual user.

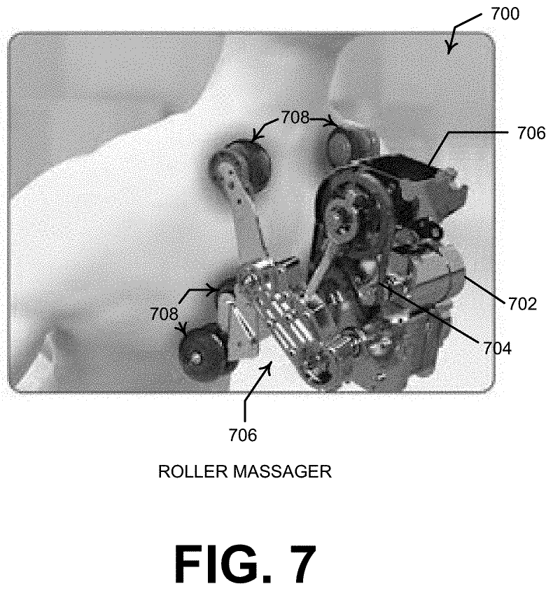

Turning now FIG. 7, the figure illustrates a roller massager 700. Roller massager 700 may be a therapeutic component of a therapeutic device and is shown with a motor 702, drive component 704, which may be a belt or a chain, or the like, and an articulating mechanism 706 for moving rollers 708 according to therapeutic parameter values of a therapy program. The roller massager 700 may also part of a massage chair or may be a standalone therapeutic device. According to description in reference to previous figures, motor 702, drive 704, and mechanism 706 may be collectively referred to as a therapy-delivering component actuator, and rollers 708 may be referred to as a therapy delivering component. Therapeutic parameters values, whether default or modified, may be used to control speed of motor 702 and movement of mechanism 706, which speed and movement would result in the therapeutic action of rollers 708 on the user's back as shown in the figure.

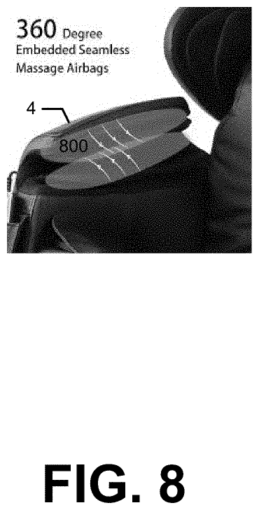

Turning now to FIG. 8, the figure illustrates seamless air bag massagers 800 that are shown applied by massage chair 4 to shoulders, or to a location where a user's shoulders would be located while receiving a therapy session in the massage chair. Massagers 800 may be examples of therapeutic-delivering components. An air source (not shown) controlled according to therapeutic parameter values may be referred to as a therapeutic component actuator.

What is claimed is:

1. A system, comprising:
a therapeutic device that includes:
one or more therapy-delivering components operated by one or more corresponding therapy-delivering component actuators;
one or more biometric parameter sensors;
a control module communicatively coupled with the one or more therapy-delivering component actuators and communicatively coupled with the one or more biometric parameter sensors;
a display interface in communication with the control module; and
a processor in communication with the control module to receive a selection of a predetermined therapeutic program, wherein the predetermined therapeutic program includes one or more default therapeutic parameter values that correspond to operation of the one or more therapy-delivering components, and wherein the processor in communication with the control module is further to modify one or more of the default therapeutic parameter values into one or more modified therapeutic parameter values based on signals produced by the one or more biometric parameter sensors that are interfaced with a particular user during a therapy session that the particular user uses the therapeutic device such that the one or more modified therapeutic parameter values differs from a corresponding one or more of the default therapeutic parameter values based on evaluation of at least one of one or more biometric parameter values derived from one or more signals from at least one of the one or more biometric parameter sensors during the therapy session.

2. The system of claim 1 wherein the display interface and the processor are part of a user device that is not attached to the therapeutic device and wherein the user device and the control module of the therapeutic device communicate via a wireless link.

3. The system of claim 1 wherein the display interface and the processor are part of the therapeutic device.

4. The system of claim 1 wherein the therapeutic device is a massage chair.

5. The system of claim 1 wherein the processor automatically selects the first predetermined therapeutic program based on a unique identifier of the particular user.

6. The system of claim 1 wherein the processor modifies the one or more of the default therapeutic parameter values into modified therapeutic parameter values based on signals received from the one or more biometric parameter sensors during a training period of the therapy session.

7. The system of claim 6 wherein the therapy session that includes the training period is a first therapy session that the particular user has used the therapeutic device.

8. The system of claim 6 wherein the therapy session that includes the training period is not a first therapy session that the particular user has used the therapeutic device.

9. The system of claim 1 wherein the modified therapeutic parameter values are stored remotely from the therapeutic device.

10. The system of claim 1 wherein the modified therapeutic parameter values are stored in a user device that communicates via wireless link with the control module.

11. The system of claim 1 wherein the modified therapeutic parameter values are stored at a therapeutic device data store at a server that is remote from the therapeutic device.

12. The system of claim 1 wherein a set of one or more default therapeutic parameter values of the first predetermined therapeutic program are selected by the processor based on a unique identifier corresponding to the particular user and based at least in part on medical history information corresponding the particular user's unique identifier.

13. The system of claim 8 wherein a set of modified therapeutic parameter values determined during a training period of a previous therapy session for the particular user are used as default therapeutic parameter values for a current therapy session for the particular user, such that the modified therapeutic parameter values as modified during the previous therapy session are further modified into new modified therapeutic parameter values during a training period of the current therapy session.

14. The system of claim 1 wherein the processor modifies a therapeutic parameter value into a modified therapeutic parameter value based on applying a correction factor to at least one of the signals received from the one or more biometric parameter sensors.

15. The system of claim 14 wherein the processor determines a correction factor to apply to one of the signals received from the one or more biometric parameter sensors based at least on one of: environmental noise level, environment temperature, barometric pressure, a number of individuals proximate the therapeutic device, time of day, or time of year.

16. A method, comprising,
based on a unique identifier, launching a therapeutic program that operates therapeutic components of a therapeutic device, wherein the therapeutic program includes one or more therapeutic parameter values that are associated with the unique identifier and that are used by the therapeutic program to control one or more of the therapeutic components, wherein the unique identifier corresponds to a particular user of the therapeutic device;
receiving one or more current biometric parameter values generated during a therapy session that the therapeutic device provides therapy to the particular user, wherein the one or more biometric parameter values are derived from corresponding one or more biometric parameter sensor signals that are generated during the therapy session based on one or more of the individual user's physiological responses during the therapy session;
comparing the one or more biometric parameter values to predetermined criteria;
in response to the comparing of the one or more biometric parameter values to the predetermined criteria, modifying the one or more therapeutic parameter values into modified therapeutic parameter values during the therapy session until current biometric parameter values satisfy the predetermined criteria; and
operating the therapeutic device according to the modified therapeutic parameter values.

17. The method of claim 16 wherein the comparing the one or more biometric parameter values to predetermined criteria and the modifying the one or more therapeutic parameter values into modified therapeutic parameter values are iteratively performed during a training period that ends when the biometric parameter values satisfy the predetermined criteria.

18. The method of claim 16 wherein the steps thereof are performed by a processor of a device that is in wireless communication with the therapeutic device.

19. The method of claim 16 wherein the sensors that generate the biometric parameter sensor signals are part of a device that is in wireless communication with the therapeutic device.

20. A massage chair, comprising:
one or more therapy-delivering components operated by one or more corresponding therapy-delivering component actuators; and
a processor to:
receive a selection of a first predetermined therapeutic program, wherein the first predetermined therapeutic program includes one or more default therapeutic parameter values that correspond to operation of the one or more therapy-delivering actuators,
modify one or more of the default therapeutic parameter values into modified therapeutic parameter values based on biometric parameter values that are derived from biometric parameter signals received from one or more biometric parameter sensors that are interfaced with a particular user during a therapy session such that at least one of the therapeutic parameter values is modified during a training period of the therapy session based on at least one of the biometric parameter values received during the training period; and
operate during a period that is not the training period the one or more therapy-delivering component actuators according to the modified therapeutic parameter values.
